(19) United States
(12) Patent Application Publication
(10) Pub. No.: US 2024/0399730 A1
(43) Pub. Date: Dec. 5, 2024

(54) METHODS OF BONDING THAT EMPLOY PREHEATING

(71) Applicant: The Procter & Gamble Company, Cincinnati, OH (US)

(72) Inventors: Todd Douglas LENSER, Liberty Township, OH (US); Randall Allen MYERS, Fairfield, OH (US); Joseph Leslie GROLMES, Madeira, OH (US)

(21) Appl. No.: 18/677,957

(22) Filed: May 30, 2024

Related U.S. Application Data
(60) Provisional application No. 63/470,188, filed on Jun. 1, 2023.

(51) Int. Cl. B32B 37/10; B32B 3/30; B32B 5/02; B32B 5/26; B32B 27/08; B32B 27/30; B32B 27/32; B32B 37/06

(52) U.S. Cl. CPC B32B 37/10; B32B 3/30; B32B 5/022; B32B 5/266; B32B 27/08; B32B 27/302; B32B 27/32; B32B 37/06; B32B 2262/0253; B32B 2305/18; B32B 2307/51; B32B 2309/02; B32B 2309/04; B32B 2310/028

(57) ABSTRACT

Methods and devices for bonding a plurality of substrates in a nip provided between an anvil and a bonding device may involve preheating a portion of a first substrate for a preheating duration to impart a preheated temperature, and then conveying the plurality of substrates to the nip to form a bond. The portion of the first substrate may reach the nip at a final temperature that is within 0° C. to 40° C. of the preheated temperature. A time-in-nip to create the bond may be less than 20 milliseconds. The preheating duration may be at least 200% longer than the time-in-nip. Laminate materials formed via such methods and devices may include unbonded areas in the nonwoven material that may have at least 5 fibers per square inch that are fused at fiber-to-fiber intersections caused by preheating the nonwoven material.

### CROSS REFERENCE TO RELATED APPLICATION

This application claims the benefit under 35 U.S.C. § 119(e) of U.S. Provisional Patent Application No. 63/470,188, filed on Jun. 1, 2023, the entire disclosure of which is incorporated herein by reference.

### FIELD

The present disclosure relates generally to bonding methods and apparatuses and more specifically to bonding methods and apparatuses that employ preheating. The present disclosure also relates to substrates and laminates with bonds formed with preheating.

### BACKGROUND

A disposable absorbent article, such as a diaper, pant, adult incontinence product, or feminine hygiene pad, may commonly be assembled from a plurality of substrates. These substrates may be bonded together to form the article. Bonding methods common in the industry may include hot melt adhesives bonds, adhesive bonds, compression/crimp bonds, thermal bonds, ultrasonic bonds, or combinations of such methods. Hot melt adhesive bonds are common, as various commercially available hot melt adhesives are available and suitable for various use cases. Where hot melt adhesive bonds are used, a secondary bond may be used in parallel or as an adjunct. A hot melt adhesive bond may provide a strong peak strength but may fail in extended use due to creep failures. A hot melt adhesive bond may add considerable capital cost for supporting equipment. Hot melt adhesives may add considerable product cost, and result in unnecessary consumption of plastics. Compression or crimp bonds may provide a strong bond but may require secondary bonds such as hot melt adhesives to support all loading cases upon the joint. Compression or crimp bonds are often suited for only small pattern areas, due to the very high contact forces and stresses, which may be over 100 MPa. Such high forces may drive high costs in machine design. Compression or crimp bonds may be sensitive to raw material choice and may be limiting to product design and/or raw material sourcing. The allowable process window in which compression or crimp bonds can function may be quite small and the process window may have a minimum substrate surface speed to achieve a sufficient bond strength. Compression or crimp bonding may be sensitive to pattern design, and patterns with a high degree of area balance may be required, further limiting the product and process design. Thermal bonding, via melting a portion of a substrate, is possible but highly material dependent. An example of thermal bonding is heated calendar rolls commonly used to bond batts of fibers into a nonwoven web. Through air bonding, where a hot air blast flows through an air permeable substrate, is another example of thermal bonding.

Ultrasonic bonding provides a strong bond. Ultrasonic bonding may provide the benefit of reducing or eliminating hot melt adhesive consumption and cost. Ultrasonic bonding may provide a potential for a larger pattern area than compression bonding. Ultrasonic bonding may enable a larger width of pattern region than compression bonding. Ultrasonic bonding may be more costly than other bonding forms, as special, high capital cost equipment is required as an upfront investment. Ultrasonic bonding may provide a softer bond aesthetic than other bonding forms. A problem with ultrasonic bonding is that it is commonly surface speed limited. As the velocity of a substrate though the working region of the sonotrode-anvil nip increases, the frequency of the sonotrode remains constant. Thus, higher substrate speeds result in fewer compression cycles from the ultrasonic sonotrode against the material of each individual bond site during the limited in-nip residence time. Increasing compression force of the sonotrode against the anvil may help, but each sonotrode is typically limited in available force and power draw. Increasing sonotrode oscillation frequency enables more compression cycles during the in-nip residence time, but the higher frequency waves may have lower energy and transfer less energy to the substrate. Said differently, ultrasonic bonding units may operate at maximum power, and increasing substrate velocity may result in less energy per unit length of substrate. Thus, ultrasonic bonding of nonwoven and film substrates is useful in the formation of disposable absorbent articles. Nevertheless, ultrasonic bonding of substrates for disposable absorbent articles have been limited in terms of process speed and/or bond strength. Additionally, commercially available ultrasonic systems had high equipment capital cost and low reliability, which further hindered research and development. Another common issue with ultrasonic and/or high compression bonding is poor bond strength, particularly at high line speeds, with high pattern areas, and/or with high pattern imbalance. For high compression bonding without ultrasonic vibration, bond strengths may be lower at lower line speeds. Heating at least one substrate may provide an increase in bond strength, line speed, and/or process window. Heating a substrate such as a thermoplastic, may soften a substrate. When compressed, a softened substrate may flow from a bond site region to an extrudate region peripheral to a bond site more easily.

Therefore, a need exists for bonding methods, techniques, and apparatuses exhibiting improved process speed and/or bond strength.

The discussion of shortcomings and needs existing in the field prior to the present disclosure is in no way an admission that such shortcomings and needs were recognized by those skilled in the art prior to the present disclosure.

### SUMMARY

Various embodiments solve the above-mentioned problems and provide methods and apparatuses useful for bonding with improved process speed and/or bond strength by preheating substrates to be bonded using a variety of devices and apparatuses.

Various embodiments relate to a method for bonding a plurality of substrates in a nip provided between an anvil and a bonding device. The method may comprise preheating a portion of a first substrate of the plurality of substrates for a preheating duration to impart a preheated temperature to the portion, and then conveying the plurality of substrates to the nip to form a bond in at least some of the plurality of substrates. The preheated portion of the first substrate may reach the nip at a final temperature that is within 0° C. to 40° C. of the preheated temperature. A time-in-nip to create the bond may be less than 20 milliseconds. The preheating duration may be at least 200% longer than the time-in-nip.

Various embodiments relate to a laminate material, such as a bonded substrate, for an absorbent article. The laminate material may comprise a non-air-through bonded nonwoven material comprising fibers, a substrate in a facing relationship with the nonwoven material, and a plurality of bonds joining the nonwoven material to the substrate. The bonds may each form a bond area in the nonwoven material and the substrate. Unbonded areas may be formed in the nonwoven material and the substrate that free of the plurality of bonds. The unbonded area in the nonwoven material may have at least 5 fibers per square inch that are fused at fiber-to-fiber intersections caused by preheating the nonwoven material.

These and other features, aspects, and advantages of various embodiments will become better understood with reference to the following description, figures, and claims.

### BRIEF DESCRIPTION OF THE DRAWINGS

Many aspects of this disclosure can be better understood with reference to the following figures.

FIG. 1 is a schematic process flow diagram illustrating a method and an apparatus for bonding a plurality of substrates that includes preheating a first substrate of the plurality of substrates.

FIG. 2 is a schematic process flow diagram illustrating a method and an apparatus for bonding a plurality of substrates that includes multi-stage preheating a first substrate of the plurality of substrates.

FIG. 3 is a schematic process flow diagram illustrating a method and an apparatus for bonding a plurality of substrates that includes preheating a first substrate and a second substrate of the plurality of substrates.

FIG. 4 is a schematic process flow diagram illustrating a method and an apparatus for bonding a plurality of substrates that includes multi-stage preheating a first substrate and a second substrate of the plurality of substrates.

FIG. 5 is a schematic process flow diagram illustrating a method and an apparatus for bonding a plurality of substrates that includes multi-surface preheating a first substrate and a second substrate of the plurality of substrates.

FIG. 6 is a schematic process flow diagram illustrating a method and an apparatus for bonding a plurality of substrates that includes preheating a first substrate and a second substrate of the plurality of substrates.

FIG. 7 is a schematic process flow diagram illustrating a method and an apparatus for bonding a plurality of substrates that includes multi-stage preheating a first substrate of the plurality of substrates and recirculation of a heated fluid.

FIG. 8 is a schematic process flow diagram illustrating a method and an apparatus for bonding a plurality of substrates that includes multi-surface preheating a first substrate and a second substrate of the plurality of substrates and recirculation of a heated fluid.

FIG. 9 is a schematic process flow diagram illustrating a method and an apparatus for bonding a plurality of substrates that includes preheating a first substrate, a second substrate, and a third substrate.

FIG. 10 is a schematic process flow diagram illustrating a method and an apparatus for bonding a plurality of substrates that includes preheating a first substrate, a second substrate, and a third substrate.

FIG. 11 is a schematic process flow diagram illustrating a method and an apparatus for bonding a plurality of substrates that includes preheating a first substrate of the plurality of substrates employing a heat shield and a cross directional flow of heated fluid.

FIG. 12 is a schematic process flow diagram illustrating a method and an apparatus for bonding a plurality of substrates that includes preheating a first substrate of the plurality of substrates employing a cross directional flow of heated fluid that is redirected in a counter machine direction.

FIG. 13 is a schematic process flow diagram illustrating a method and an apparatus for bonding a plurality of substrates as well as details of the bonded substrate.

FIG. 14 is a schematic diagram illustrating a bonding device having bearer rings on a surface thereof.

FIG. 15 is a chart plotting temperature vs. time for a single fiber in a nonwoven due to forced convection preheat or natural convection cooling.

FIG. 16 is a chart plotting heat exchanger length for various web speeds and pre-heat air temperatures for a convective heat transfer embodiment.

FIG. 17 is a chart plotting cooling length for various web speeds and final temperatures at various representative cooling rate after a 120° C. preheat.

FIG. 18 is a schematic process flow diagram illustrating a method and an device for bonding and preheating.

FIG. 19 is a schematic diagram illustrating a various preheating zones for a back ear of a diaper.

FIG. 20 is an elastic modulus response curve of a representative PP over range of temperatures and crystallinity.

FIG. 21 is an example of Differential Scanning Calorimetry (DSC) results for determination of melting range.

It should be understood that the various embodiments are not limited to the examples illustrated in the figures.

### DETAILED DESCRIPTION

### Introduction and Definitions

This disclosure is written to describe the invention to a person having ordinary skill in the art, who will understand that this disclosure is not limited to the specific examples or embodiments described. The examples and embodiments are single instances of the invention which will make a much larger scope apparent to the person having ordinary skill in the art. Unless defined otherwise, all technical and scientific terms used herein have the same meaning as commonly understood by the person having ordinary skill in the art. It is also to be understood that the terminology used herein is for the purpose of describing examples and embodiments only, and is not intended to be limiting, since the scope of the present disclosure will be limited only by the appended claims.

All the features disclosed in this specification (including any accompanying claims, abstract, and drawings) may be replaced by alternative features serving the same, equivalent, or similar purpose, unless expressly stated otherwise. Thus, unless expressly stated otherwise, each feature disclosed is one example only of a generic series of equivalent or similar features. The examples and embodiments described herein are for illustrative purposes only and that various modifications or changes in light thereof will be suggested to the person having ordinary skill in the art and are to be included within the spirit and purview of this application. Many variations and modifications may be made to the embodiments of the disclosure without departing substantially from the spirit and principles of the disclosure. All such modifications and variations are intended to be included herein within the scope of this disclosure. For example, unless otherwise indicated, the present disclosure is not limited to particular materials, reagents, reaction materials, manufacturing processes, or the like, as such can vary. It is also to be understood that the terminology used herein is for purposes of describing particular embodiments only and is not intended to be limiting. It is also possible in the present disclosure that steps can be executed in different sequence where this is logically possible.

All numeric values are herein assumed to be modified by the term “about,” whether or not explicitly indicated. The term “about” generally refers to a range of numbers that one of skill in the art would consider equivalent to the recited value (for example, having the same function or result). In many instances, the term “about” may include numbers that are rounded to the nearest significant figure.

In everyday usage, indefinite articles (like “a” or “an”) precede countable nouns and noncountable nouns almost never take indefinite articles. It must be noted, therefore, that, as used in this specification and in the claims that follow, the singular forms “a,” “an,” and “the” include plural referents unless the context clearly dictates otherwise. Thus, for example, reference to “a support” includes a plurality of supports. Particularly when a single countable noun is listed as an element in a claim, this specification will generally use a phrase such as “a single.” For example, “a single support.” Where a range of values is provided, it is understood that each intervening value, to the tenth of the unit of the lower limit (unless the context clearly dictates otherwise), between the upper and lower limit of that range, and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

In this specification and in the claims that follow, reference will be made to a number of terms that shall be defined to have the following meanings unless a contrary intention is apparent.

“Disposed on” or “Disposed” refers to a positional state indicating that one object or material is arranged in a position adjacent to the position of another object or material. The term does not require or exclude the presence of intervening objects, materials, or layers.

“Absorbent article” refers to devices that absorb and contain liquid, and more specifically, refers to devices that are placed against or in proximity to the body of the wearer to absorb and to contain various exudates discharged from the body.

“Align” or “aligned” or “aligning” means to place or to arrange in a straight line. Aligning edges of substrates, therefore, means arranging the substrates so that the edges in question extend along approximately the same line. It is to be appreciated that aligning edges of substrates can be accomplished in a variety of ways, including placing the substrates one on top of the other or side by side.

“Facing relationship” refers to a relative positioning of materials, such as substrates, in which a surface of one material is oriented toward a surface of another material. For example, when two substrates are stacked on top of each other, they are in a facing relationship. The term does not require or exclude the presence of intervening objects, materials, or layers.

“Machine direction” (MD) refers to the direction of material flow through a process. In addition, relative placement and movement of material can be described as flowing in the machine direction through a process from upstream in the process to downstream in the process.

“Cross direction” (CD) refers to a direction that is generally perpendicular to the machine direction.

Heating a substrate may lower the modulus of the substrate. As the modulus of most materials decreases with increasing temperature, the required force from a compression bonding unit such as an ultrasonic sonotrode or a crimping tool to cause flow from the nip region of a bond site to an extruded material region outside the bond site may decrease. Such bonds may occur in a membrane region corresponding to a compression nub and/or an extrudate region of material forced to flow out of a nub region during the bonding process. In some embodiments, substrates may bond when a portion of at least one of the substrates undergoes a phase transition from a solid phase to a liquid phase and said substrates flow together. Heating substrates with hot air, or other fluid, to a temperature below their melting temperature may substantially increase bond strength at a given speed and/or may enable high quality bonds at higher line speeds. Such methods may be particularly useful in the conversion of slow, high cost online processes on multiple converting lines to a single pre-processor line. For example, instead of a complex back ear laminator or back ear and fastener laminator on multiple absorbent article lines, a single pre-processing line may make completed back ears with fastening tabs for several absorbent article lines. Such machines are known in the industry, but are often line rate limited to about 300 m/min to about 400 m/min. By pre-heating substrates, the line rate of existing laminators may be increased substantially. Higher throughput from a given capital asset may result in significant capital and operating cost reductions.

Merely heating a substrate may be insufficient to provide a benefit to a converting line. The substrate may be heated to a controlled temperature throughout the entire fiber and/or film thickness. The substrate may be maintained at a temperature near the preheat temperature until and through the bonding process. The relatively low basis weight webs, small nonwoven fiber thicknesses, thin films, and relatively high line speeds common to the absorbent article industry led to substrates which heat and cool surprisingly quickly. For example, cooling of about 25° C. in 50 mm MD and heating of over 30° C. in 50 mm MD has been observed. A simple nonwoven heated to 115° C. may cool off to about 740 within less than 100 mm at 450m/min surface speed.

Another complication is that many substrates are susceptible to damage from heating. For example, bicomponent nonwovens may curl or shrink when heated, as a first region of the fiber contracts or expands more than a second region of a fiber when heated. Examples of bicomponent fibers may include polyethylene (PE), polypropylene (PP), and/or polyethylene terephthalate (PET), including combinations such as PE/PP, PP/PET, and PP/PP. For PP/PP, the polypropylene may have different forms, such as a metallocene form in a first region and a Ziegler-Natta form in a second region. Bicomponent fibers may be a sheath and core, side-by side, eccentric, islands in the sea, or other arrangement of regions of different material properties. Such shrinkage may cause wrinkles, holes, or fold-overs in the nonwoven. Some nonwovens may partially melt and fuse at temperatures under, at, or over their melting temperature. Unintentional bonds may be formed between fibers in this manner, which may make a nonwoven feel hard. Some elastomer substrates, such as extensible films, may lose their elastomeric properties when exposed to excessive heat, especially if said substrates are extended while heated.

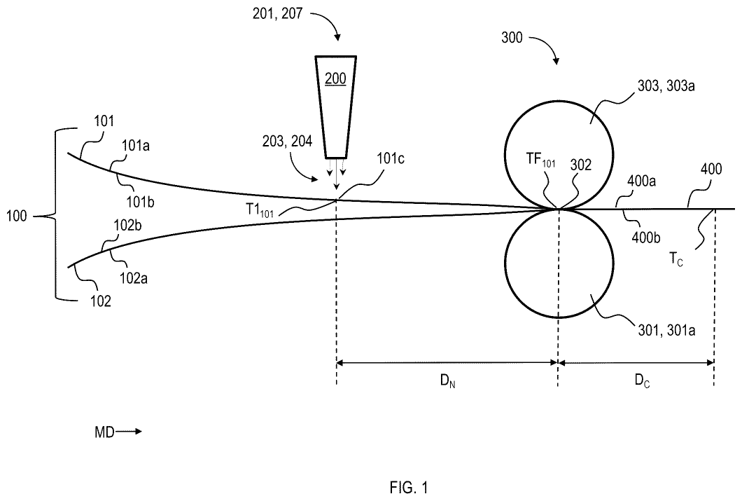

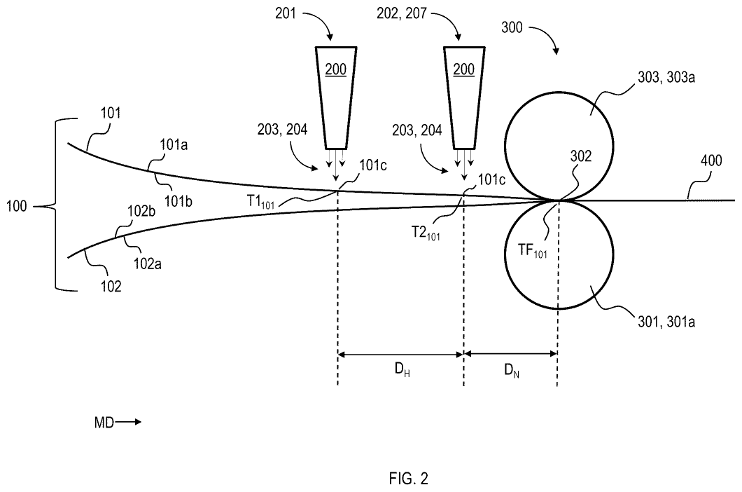

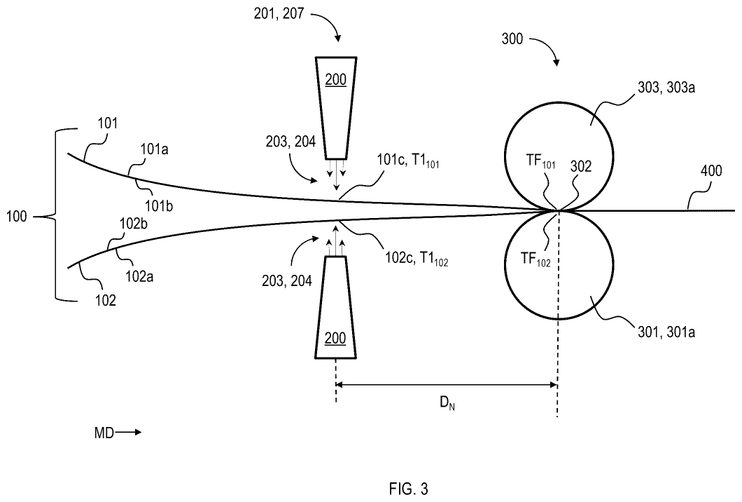

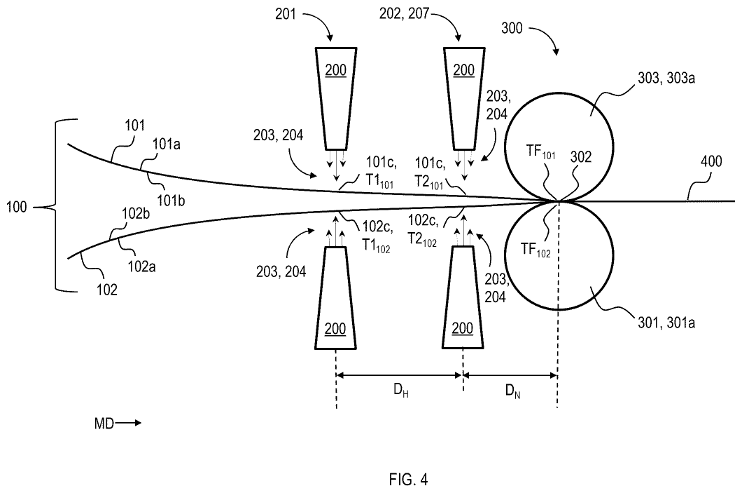

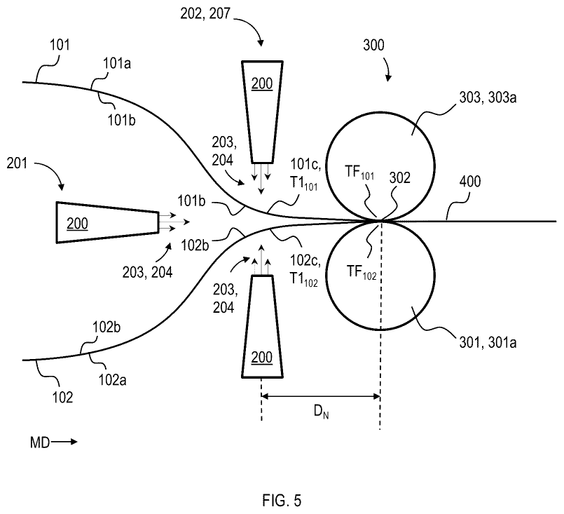

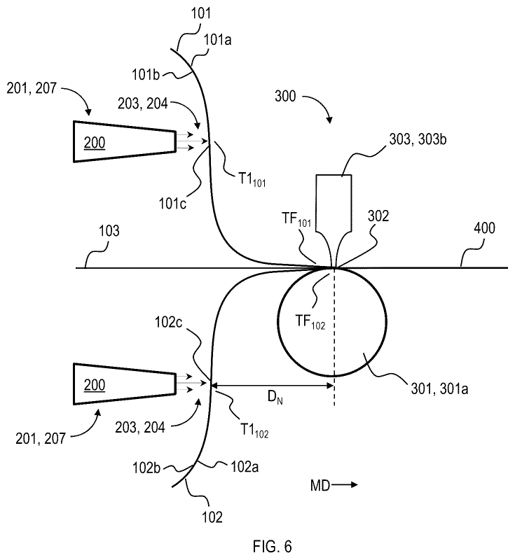

FIG. 1, FIG. 2, FIG. 3, FIG. 4, FIG. 5, and FIG. 6 cooperate to illustrate a variety of configurations for a method and an apparatus for bonding a plurality of substrates 100 that includes preheating a first substrate 101 of the plurality of substrates 100. It is to be appreciated that any number of configurations are within the scope of the present disclosure and that the specifically illustrated embodiments do not limit this disclosure. Any number of preheating stages may be employed to heat any of the plurality of substrates on any of their respective sides and from any angle.

FIG. 1 is a schematic process flow diagram illustrating a method and an apparatus for bonding a plurality of substrates 100 that includes preheating a first substrate 101 of the plurality of substrates 100. The plurality of substrates 100 may include a first substrate 101 and a second substrate 102. The first substrate 101 may include an outer portion 101a and an inner portion 101b. Similarly, the 2nd substrate 102 may include an outer portion 102a and an inner portion 102b. The inner portions 101b, 102b of the first substrate 101 and the second substrate 102 may be in a facing relationship. The plurality of substrates 100 may be conveyed in a machine direction MD toward a preheating apparatus 200 and toward a bonding machine 300. The preheating apparatus 200 may be a component of a first preheating stage 201. The preheating apparatus 200 may direct energy 203 toward one or more of the plurality of substrates 100. For example, as shown in FIG. 1, the preheating apparatus 200 may direct a heated fluid 204 toward the outer portion 101a of the first substrate 101. The energy 203, such as the heated fluid 204, may heat a heated portion 101c of the first substrate 101. The heated fluid may have a temperature in the range of about 90° C. to about 300° C., or about 100° C. to about 275° C., or about 100° C. to about 250° C. Thereby methods according to various embodiments may comprise preheating a portion of the first substrate 101 of the plurality of substrates 100 for a preheating duration to impart a preheated temperature T1₁₀₁ to the heated portion 101c.

The heated portion 101c may extend over the outer portion 101a of the first substrate 101 and also through a thickness of the first substrate 101 toward the inner portion 101b of the first substrate 101. The heated portion 101c may have a first preheated temperature T1₁₀₁. The first preheated temperature T1₁₀₁ may be an average temperature of the first heated portion 101c or maybe a surface temperature of the first substrate 101 measured on either the outer portion 101a or the inner portion 101b. The heated portion 101c of the first substrate 101 may comprise a substantial portion of a thickness thereof.

The plurality of substrates 100 may pass beyond the first preheating stage 201 toward the bonding machine 300. The bonding machine 300 may comprise an anvil 301 and a bonding device 303. According to various embodiments the anvil 301 and/or the bonding device 303 may be heated. The anvil 301 may be a first drum 301a. The bonding device 303 maybe a second drum 303a. A nip 302 may be formed between the anvil 301 and the bonding device 303. The plurality of substrates 100 may be conveyed through the nip 302 where pressure and/or energy, such as ultrasonic vibrational energy, may form one or more bonds between the plurality of substrates to form a bonded substrate 400. A bond, so formed, may be free of adhesives. A time-in-nip to create the bond may be less than 20 milliseconds, preferably less than 10 milliseconds, more preferably less than 5 milliseconds. According to various embodiments, conveying the plurality of substrates 100 through the nip 302 may occur at a speed greater than 250 m/min, or greater than 300 m/min, or greater than 400 m/min.

Preheating one or more of the plurality of substrates 100 may serve to facilitate stronger bonds between the plurality of substrates 100. It is to be appreciated that while multiple preheating stages may be employed as will be discussed hereinafter, the first preheating stage 201 may be the only preheating stage and may therefore be the final preheating stage 207. In any case, the heated portion 101c of the first substrate 101 may cool as it is conveyed a distance D_{N} from a final preheating stage 207 to the nip 302. According to various embodiments, preheating the heated portion 101c of the first substrate 101 may be completed within a distance D_{N} of about 5 mm to about 500 mm, or about 5 mm to about 400 mm, or about 5 mm to about 300 mm. It may therefore be advantageous to reduce the distance D_{N} between the final preheating stage 207 and the nip 302 in order to increase the final preheated temperature TF₁₀₁ of the heated portion 101c of the first substrate 101 at the nip 302. Like the first preheated temperature T1₁₀₁, the final preheated temperature TF₁₀₁ may be an average temperature of the first heated portion 101c or maybe a surface temperature of the first substrate 101 measured on either the outer portion 101a or the inner portion 101b. After exiting the nip 302 the bonded substrate 400 may travel a distance Dc before it reaches a cooled temperature Tc. The cooled temperature Tc may be an average temperature of the bonded substrate 400 or maybe a surface temperature of the first substrate 101 measured on either the top surface 400a or the bottom surface 400b. The heated portion 101c of the first substrate 101 may reach the nip at a final preheated temperature TF₁₀₁ that is within 0° C. to 40° C. of first preheated temperature T1₁₀₁. The total preheating duration may be at least 200% longer than the time-in-nip, or at least 300% longer than the time-in-nip, or at least 400% longer than the time-in-nip, or at least 500% longer than the time-in-nip. According to various embodiments, the preheating duration may be about 50 milliseconds to about 200 milliseconds.

Testing has determined certain films may experience thermal degradation between 50° C. and 100° C. Some nonwovens may be safely heated to 90-160° C. In some embodiments, the nonwovens may be heated individually at a location proximate to a point at which they are combined into a nonwoven-elastomeric-nonwoven structure on a vacuum anvil. The laying on of substrates may be proximate the bonding nip, with ultrasonic bonding immediately after. The CD spreading of the elastomeric film may be displaced from the laying on by an intermediate transfer roll. The roll may be provisioned with vacuum and/or nubs to maintain the extensible film in a CD extended state. Where a plurality of canted disk spreader ring pairs are utilized for this purpose, the disk pairs may be arranged adjacent each other in the CD. The spreader pairs may be driven from an infeed roller or from the vacuum transfer roller. The canted disks may be maintained in a preferred angular disposition by a four, six, or other mechanical linkage, which may include threaded rods or left hand/right hand threaded rod elements. Such a mechanism may maintain symmetry. One of the substrates may at least partially wrap a blade or rotary or star-converter style sonotrode prior to bonding, which may reduce heat transfer from a preheated substrate to a temperature sensitive intermediate layer substrate, which may be an styrenic block copolymer film. In such case, a first nonwoven at first temperature, a second nonwoven at second temperature, and a film or stranded elastomeric at third temperature may be laminated. The third temperature may be lower than, or different than, the first temperature and/or the second temperature. The first temperature and the second temperature may be chosen along with a preheating distance for the first nonwoven and the second nonwoven, respectively, such that the second temperature is largely unaffected by the limited contact time of the first nonwoven and/or against the elastomeric substrate. The first nonwoven and/or the second nonwoven may be hotter than the film, such that a good bond strength is produced without adverse effect on the film. Said differently, there may be insufficient time and heat transfer rate for the film to heat up and be damaged. There may be insufficient time and heat transfer rate for the nonwovens to cool substantially before bonding. An elastomeric film, which may be held extended in a CD stretched state, in such as bonding stack may be displaced outside of nonwoven-nonwoven bond site. The net bonding is thus simple nonwoven-nonwoven bonding through a hole in the film.

Preheating as a process aide may be applicable to any configuration of bonding, such as a using a sonotrode or an ultrasonic horn. Rotary sonotrodes may have reduced ultrasound amplitude and may benefit greatly from the softening of substrates due to preheating. Traditional rotary sonotrodes are limited in CD width. An example width may be about 85 mm wide per horn for a Herrmann Ultrasonics RSD 20-85 rotary sonotrode unit. Blade style sonotrodes used in nonwovens are limited as well, with commercial sizes including 161 mm, 270 mm, and 320 mm CD width among the largest. Unlike rotary sonotrodes, blade style sonotrodes may be arranged in a broad front configuration via a nesting approach. In addition to these traditional sonotrodes, substrate preheat may be useful for wide rotary sonotrodes, where a plurality of sonotrodes excite a wide rotary sonotrode, which may be 1-2m wide in CD. Preheating may work for sonotrodes which excite an amplitude wave in a direction orthogonal to a planar web. Preheating may work for sonotrodes which exhibit a diametral amplitude expansion and contraction. Substrate preheating may aide sonotrodes which exhibit an amplitude wave traverse to the direction of movement of the substrate. The latter may include a sonotrode which shears across a CD direction of a substrate. Preheating may aid ultrasonic bonding for sonotrodes which exhibit a plurality of excitation modes. For example, star converters may superimpose two or more standing waves to develop a nominally uniform diametral expansion. Other forms of sonotrodes may superimpose a lateral displacement excitation mode with a diametral expansion and contraction excitation mode. Substrate preheating may be applicable to any sonotrode where a vibrational mode is excited in a mechanical element and used to impart a force on a substrate. Vibrational mode is used here in the contact of a natural resonance shape corresponding to an eigenvalue of the mechanical system.

Preheating may be useful for bonding of thermoplastics to other thermoplastics. Preheating may also be useful for a thermoplastic bonded to a non-thermoplastic substrate. Preheating may aid bond strength in bonding a thermoplastic to a cellulosic material or a thermoset material. Preheating may further aide bonding of a nonwoven substrate, a film substrate, and/or a laminate substrate comprising both nonwoven and film layers. Preheating may aid bonding of cellulosic material to cellulosic material via an ultrasonic bond with added water.

In rotary or star sonotrode rotary embodiments, the sonotrode and/or anvil may be driven at a speed different than the surface velocity of the substrate. In rotary or star sonotrode rotary embodiments, the sonotrode may be driven at a speed different than the surface velocity of an anvil. In blade style ultrasonics, the anvil may be driven at a speed different than the surface speed of a substrate. In compression bonding, a nub roll and/or an anvil roll may be driven at a speed different than the substrate. In compression bonding, a nub roll may be driven at a surface speed different than the anvil roll surface speed. Such speed differences may be defined by overhang or underhang of a nub relative to a bearer ring.

Such speed differences may be an overspeed or an underspeed. An overspeed may be one component running faster than a mating component. An underspeed may be one component running slower than a mating component. An overspeed may be one component running faster than a substrate. An underspeed may be one component running slower than a substrate. Overspeed may be +5%, +10%,+20%,+30%, +40% or more. Overspeed may draw material into a bonding nip. Overspeed may smear a material. Underspeed may be −5%, −10%, −20%, −30%, −40% or more. Underspeed may smear a material. Underspeed may reduce localized basis weight variation in a material. Underspeed may create an accumulation of material upstream of a sonotrode or another bonding element. Such accumulation of material may be extruded to a periphery of a bond site. Such extrudate may provide a significant portion of the bond strength of an individual nub.

An advantage of modem sonotrodes is a higher force capacity. For example, modem rotary sonotrodes may have force ratings of at least 3 kN, 4 kN, 5 kN, 6 kN, or higher per 85 mm of substrate CD. Such load ratings may be due to having an arrangement with bearing supports on both first and second sides of a sonotrode. Older rotary sonotrodes may be a cantilevered trumpet shape in cross-section. Modern devices may essentially be a mirrored image of such a trumpet shape.

The combination of high force, ultrasonic amplitude, high line speed, and/or substrate preheat may yield improved bonding results beyond the effect of any one process variable. High force may drive material flow. High line speed may provide a kinematic compression improving bonding. Ultrasonic amplitude may provide improve bonding. Substrate preheat may reduce modulus. Shear between a blade sonotrode, a mismatched speed rotary sonotrode, a mismatched speed compression nub or anvil may further improve bonding. Preheating may reduce surface oxidation of polymers, which may otherwise inhibit bonding.

Suitable heaters may comprise Leister (Switzerland) heaters LE 10,000, Mistral, or Hotwind product lines, or Tutco Sureheat Max 6 kW. Suitable fans may be any fan or blower of suitable volumetric flow rate. Where hot air recirculation is utilized, a fan suitable for high temperature service such as Leister RBR or Leister Whisper models may be used. Thermocouples, RTD's (resistance temperature devices) or temperature sensors may be utilized. A programmable logic controller (PLC) or standalone temperature controller may be utilized to control temperature. Such controller may comprise a feedback control loop with a pulse width modulated or variable current output. Temperature feedback may be from an internal heater stage, heater exit, air in a duct, nozzle or heatbox downstream of the heater, and/or by direct measurement of substrate temperature. Temperature measurement of air in the recirculation loop may be used by the controller. Controller output may be preprogrammed to vary with any of the temperature readings, line speed, a mathematical model of the process, time, and/or airflow. Air velocity and/or mass flow rate may be used in control of heating. Airflow rate may be controlled by a variable frequency drive or other variable speed drive for the fan or blower element. Heaters may heat air to one or a plurality of nozzles. Fans or blowers may supply air to one or a plurality of nozzles.

FIG. 2 is a schematic process flow diagram illustrating a method and an apparatus for bonding a plurality of substrates 100 that includes multi-stage preheating a first substrate 101 of the plurality of substrates 100. FIG. 2 is identical to FIG. 1 except that a second preheating stage 202 has been included. The second preheating stage 202 is the final preheating stage 207 because it is closest to the nip 302 at a distance D_{N}. The total preheating distance D_{H} between the first preheating stage 201 and the second preheating stage 202 represents a distance over which the first substrate 101 of the plurality of substrates 100 is heated prior to entering the nip 302. The second preheating stage 202 heats the heated portion 101c of the first substrate 101 to a second preheated temperature T2₁₀₁, which like the other substrate temperatures T₁₀₁, TF₁₀₁, may be an average temperature of the first heated portion 101c or maybe a surface temperature of the first substrate 101 measured on either the outer portion 101a or the inner portion 101b. Put differently, the first preheating stage 201 may comprise heating the heated portion 101c of the first substrate 101 for a first duration to impart a first preheated temperature T1₁₀₁ to the heated portion 101c and the second preheating stage 202 may comprise heating the heated portion 101c of the first substrate 101 for a second duration to impart a second preheated temperature T2₁₀₁ to the heated portion 101c. The first preheated temperature T1₁₀₁ and the second preheated temperature T2₁₀₁ may be the same or different.

Heating may be in multiple stages, at a first temperature and at a second temperature. A first temperature may be higher than or different than a second temperature in some embodiments. For example, a PP which melts at about 162° C. may be heated from 22° C. to about 100-120° C. in a first step by air at 200-300° C. Due to the large temperature difference between this first stage air and the initial polymer, the web may heat very quickly, as only a partial time constant is required. In a second stage, a lower temperature air, such as 130° C., is applied to finish the heating to an intended target temperature. This second air temperature may be lower than the first stage air temperature. This second hot air may be directed adjacent one of more substrates proximate the nip point to inhibit cooling prior to bonding. In other embodiments, the initial heating may be a lower temperature. For example, a web sensitive to wrinkling during heating may be partially heated by a lower temp first heater. The web may then be heated by a higher temperature air stream proximate the nip point. Staged heating may mitigate the effects of wrinkling by the web in such a case.

The preheat temperature may be varied based on the substrate velocity. The volumetric air flow may be varied based on the substrate velocity. In the case of multi-stage heating, one or more of the temperature setpoints may be adjusted based on line speed. In an embodiment, the volumetric airflow may be held constant. The heat transfer may be partially insensitive to air velocity once turbulent flow is achieved. In an embodiment, at least one temperature is adjusted based on substrate velocity and/or line speed. Small adjustments of applied air temperature may lead to large changes in process behavior. Substrate temperature may be measured directly and used for control feedback. Air temperature from any of the heaters may be measured and used for control feedback. Where a multi-stage system is used, an embodiment may have the higher temperature heater as the speed varying setpoint. Heaters and/or airflow may be turned off or reduced at slow line speeds, such as thread. Airflow may be left on during line ramp down, to cool the heaters and equipment.

FIG. 3 is a schematic process flow diagram illustrating a method and an apparatus for bonding a plurality of substrates 100 that includes preheating a first substrate 101 and a second substrate 102 of the plurality of substrates 100. FIG. 2 is identical to FIG. 1 except that an additional preheating apparatus 200 has been added to the first preheating stage 201. The additional preheating apparatus 200 may direct energy 203 toward one or more of the plurality of substrates 100. For example, as shown in FIG. 3, the additional preheating apparatus 200 may direct a heated fluid 204 toward the outer portion 102a of the second substrate 102. The energy 203, such as the heated fluid 204, may heat a heated portion 102c of the second substrate 102 to a first temperature T₁₀₂, may be an average temperature of the heated portion 102c or maybe a surface temperature of the second substrate 102 measured on either the outer portion 102a or the inner portion 102b. After being conveyed a distance D_{N} from the first preheating stage 201 to the nip 302, the first substrate 101 may have a final preheated temperature TF₁₀₁ and the second substrate 102 may have a final preheated temperature TF₁₀₂.

FIG. 4 is a schematic process flow diagram illustrating a method and an apparatus for bonding a plurality of substrates 100 that includes multi-stage preheating a first substrate 101 and a second substrate 102 of the plurality of substrates 100. FIG. 4 is identical to FIG. 3 except that a second preheating stage 202 has been included. The second preheating stage 202 is the final preheating stage 207 because it is closest to the nip 302 at a distance D_{N}. The total preheating distance D_{H} between the first preheating stage 201 and the second preheating stage 202 represents a distance over which the first substrate 101 of the plurality of substrates 100 is heated prior to entering the nip 302. The second preheating stage 202 heats not only the heated portion 101c of the first substrate 101 but also the heated portion 102c of the second substrate 102 to a second preheated temperature T2₁₀₂. After being conveyed a distance D_{N} from the final preheating stage 207 to the nip 302, the first substrate 101 may have a final preheated temperature TF₁₀₁ and the second substrate 102 may have a final preheated temperature TF₁₀₂.

FIG. 5 is a schematic process flow diagram illustrating a method and an apparatus for bonding a plurality of substrates 100 that includes multi-surface preheating a first substrate 101 and a second substrate 102 of the plurality of substrates 100. FIG. 5 is similar to FIG. 3 and FIG. 4, except that the first preheating stage 201 is positioned to direct energy 203 in the form of a heated fluid 204 at the inner portion 101b of the first substrate 101 and at the inner portion 102b of the second substrate 102. The second preheating stage 202 is the final preheating stage 207 because it is closest to the nip 302 at a distance D_{N}. The total preheating distance D_{H} between the first preheating stage 201 and the second preheating stage 202 is not shown, since both of the heating stages 201, 202 heat the substrates 101, 102 at approximately the same distance D_{N} from the nip. The substrates 101, 102 are heated to first preheated temperatures T1₁₀₁, T1₁₀₂, which may be an average temperature of the first heated portions 101c, 102c or may be a surface temperature of the substrates 101, 102 measured on either the outer portion 101a, 102a or the inner portions 101b, 102b. After being conveyed a distance D_{N} from the final preheating stage 207 to the nip 302, the first substrate 101 may have a final preheated temperature TF₁₀₁ and the second substrate 102 may have a final preheated temperature TF₁₀₂.

FIG. 6 is a schematic process flow diagram illustrating a method and an apparatus for bonding a plurality of substrates 100 that includes preheating a first substrate 101 and a second substrate 102 of the plurality of substrates 100. A third substrate 103 is also included in the plurality of substrates 100. It is to be appreciated that although the third substrate 103 is not shown as being preheated, it may be preheated in the same variety of ways as any of the plurality of substrates 100. A first preheating stage 201 comprises a plurality of preheating apparatuses 200, which are positioned to direct energy 203 in the form of a heated fluid 204 at the inner portion 101b of the first substrate 101 and at the inner portion 102b of the second substrate 102. The inner portion 101b of the first substrate 101 is heated to a first temperature T1₁₀₁ and the inner portion 102b of the second substrate 102 is heated to a first temperature T1₁₀₂. After being conveyed a distance D_{N} from the first preheating stage 201 to the nip 302, the first substrate 101 may have a final preheated temperature TF₁₀₁ and the second substrate 102 may have a final preheated temperature TF₁₀₂.

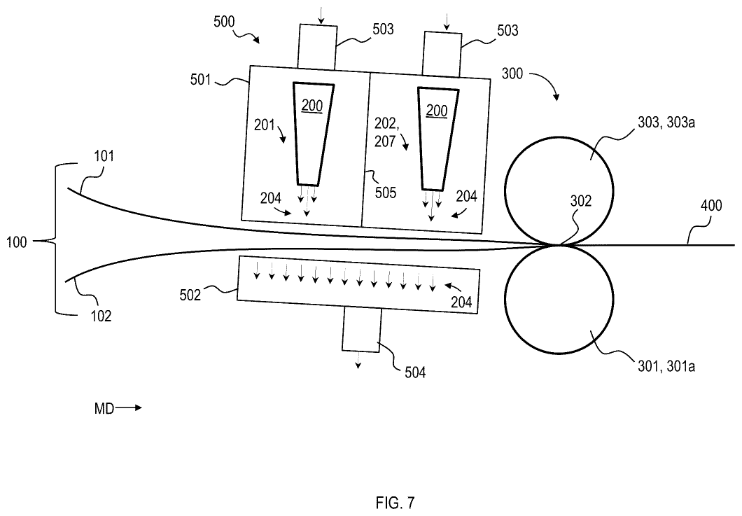

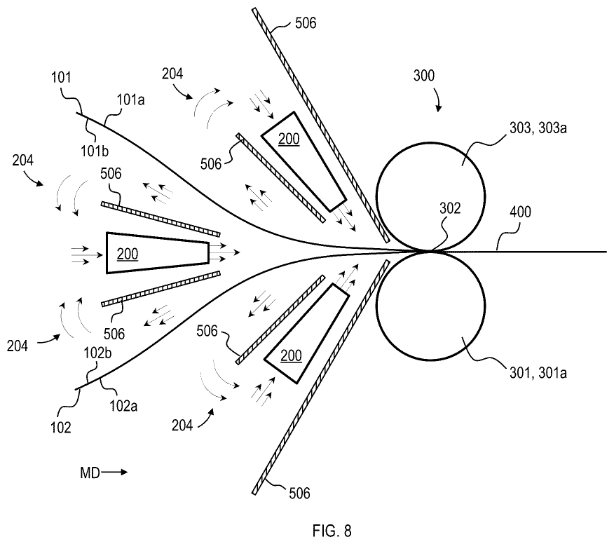

FIG. 7 and FIG. 8 cooperate to illustrate a variety of configurations for a method and an apparatus for bonding a plurality of substrates 100 that includes preheating a first substrate 101 of the plurality of substrates 100 and recirculation of a heated fluid 204. It is to be appreciated that any number of configurations are within the scope of the present disclosure and that the specifically illustrated embodiments do not limit this disclosure. Any number of preheating stages may be employed to heat any of the plurality of substrates on any of their respective sides and from any angle and many recirculation methods and apparatuses may be employed. The thermal energy to raise the temperature of a substrate may be low, and may be a small percentage, such as 10% or 20% of the energy applied to create a hot air jet. To improve the efficiency of the system, the air after heating a substrate may be directed to the inlet of a fan or flow, further ducted to a commercial hot air heater, and then ducted back into a nozzle, drum, hot air box, or other appropriate heat transfer arrangement. Operating costs and environmental impact of the system may thus be significantly reduced. The heat and the airflow may be driven via an electrical source, which may be renewably sourced. Such a system may displace hot melt adhesives and thus reduce the environmental impact of a diaper.

FIG. 7 is a schematic process flow diagram illustrating a method and an apparatus for bonding a plurality of substrates 100 that includes multi-stage preheating a first substrate 101 of the plurality of substrates 100 and recirculation of a heated fluid 204. FIG. 7 is identical to FIG. 2, except for the addition of a heat recycling system 500. The heat recycling system 500 may comprise a hot side 501 and a cool side 502. The hot side 501 may be associated with one or more preheating apparatuses 200. For example, as shown, the hot side 501 may surround a plurality of preheating apparatuses 200 or a plurality of preheating stages, such as a first preheating stage 201 and a second preheating stage 202. The preheating stages 201, 202 and/or the preheating apparatus 200 may be separated by one or more optional subdividers 505 disposed within the hot side 501 of the heat recycling system 500. The hot side 501 of the heat recycling system 500 may comprise one or more inlets 503. According to some versions, each inlet 503 may supply a fluid, such as air, to a different heating stage 201, 202 and/or to a different preheating apparatus 200, which may convert the inlet fluid to heated fluid 204. After passing through the hot side 501 of the heat recycling system 500, the heated fluid 204 may impinge upon one or more the plurality of substrates 100. The cool side 502 of the heat recycling system may be positioned to receive the heated fluid 204 after it has impinged upon or passed through one or more of the plurality of substrates 100. The cool side 502 may collect and recycle the heated fluid 204, via an outlet 504, which may redirect the heated fluid 204 to the inlet 503 to be reused, thereby conserving heat energy.

FIG. 8 is a schematic process flow diagram illustrating a method and an apparatus for bonding substrates that includes multi-surface preheating a first substrate 101 and a second substrate 102 and recirculation of a heated fluid 204. FIG. 8 is identical to FIG. 5, but for the inclusion of airflow guides 506. The airflow guides 506 may channel and/or redirect heated fluid 204. The heated fluid 204 may be redirected to flow in a direction that is substantially opposed to the machine direction MD and/or in a direction that facilitates recirculation to conserve energy. Redirecting the heated fluid 204 in a direction that is substantially opposed to the machine direction MD may allow the heated fluid 204 to pass over a surface of a substrate 101, 102 in a direction that is counter to the direction in which that substrate 101, 102 is being conveyed toward the nip 302. Such a counterflow operation allows the substrate 101, 102 to encounter hotter air as it proceeds closer to the nip, facilitating a greater degree of heat transfer from the heated fluid 204 to the substrate 101, 102 overall.

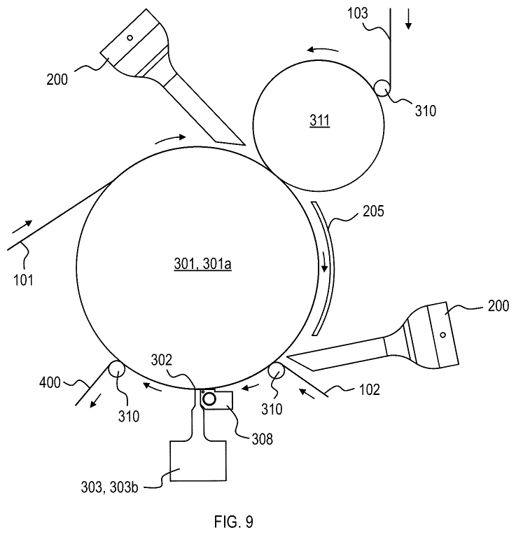

FIG. 9 is a schematic process flow diagram illustrating a method and an apparatus for bonding a plurality of substrates 100 that includes preheating a first substrate 101, a second substrate 102, and a third substrate 103. The third substrate 103 may be heated to the same or different preheat temperature than the first substrate 101 and the second substrate 102. Due to the tendency of nonwoven fibers to curl upon heating, especially bicomponent fibers, a web spreader 311 may be utilized upstream of the heating zone. Along with appropriate choice of heating temperature and nozzle configuration, this may be key to a successful process. For example, the first substrate 101 maybe conveyed about an anvil 301, which may be a first drum 301a, before passing a spreader 311 about which a third substrate 103 may be conveyed optionally after passing an idler 310.

The first substrate 101 and the third substrate 103 may be preheated by a preheating apparatus 200 and conveyed further along the anvil 301 toward a nip 302 formed between the anvil 301 and a bonding device 303 which may be an ultrasonic bonding device 303b or a second drum (not shown). A heat shield 205 may be provided along at least part of the path that the first substrate 101 and the third substrate 103 take toward the nip 302. The heat shield 205 may reduce a degree to which the first substrate 101 and the third substrate 103 cool after being preheated and prior to reaching the nip 302. A second substrate 102 may be preheated along with the first substrate 101 and the third substrate 103 as the second substrate 102 is brought into contact with the anvil 301 after passing an optional idler 310. A cooler 308 may be disposed adjacent to the bonding device 303 to prevent the Bonding device 303 from overheating. Although the cooler 308 is shown downstream of the sonotrode, it is to be appreciated that the cooler 308 may be mounted upstream of the sonotrode. The cooler of 308 may be supplied with a cooling fluid. After the plurality of substrates 100 pass through the nip 302 a bonded substrate 400 may be produced and optionally redirected via an idler 310. Such a configuration is particularly useful when the third substrate 103 comprises an elastic material to be bonded between the first substrate 101 and the second substrate 102, which may be any type of nonwoven or film material. As will be illustrated here in after the bonds in the bonded substrate 400 may be positioned so as to avoid the elastic film sandwiched between the first substrate 101 and the second substrate 102.

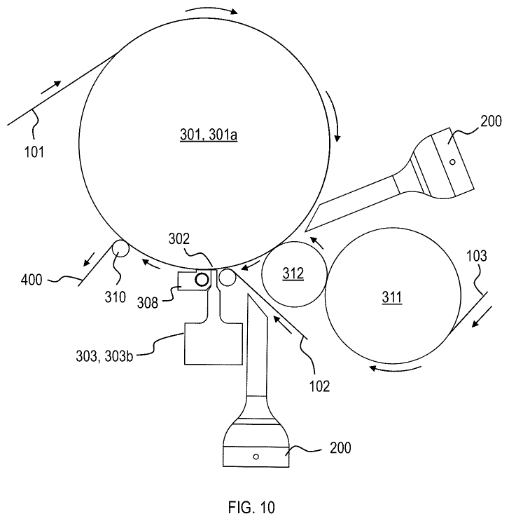

FIG. 10 is a schematic process flow diagram illustrating a method and an apparatus for bonding a plurality of substrates 100 that includes preheating a first substrate 101, a second substrate 102, and a third substrate 103. The third substrate 103 may be heated to the same or different preheat temperature than the first substrate 101 and the second substrate 102. FIG. 10 is similar to FIG. 9 except that the spreader 311 and the first heating apparatus 200 is positioned closer to the point at which the second substrate 102 is preheated, which may avoid the need for a heat shield 205. Additionally, the spreader 311 may transfer the third substrate 103 to a transfer roll 312. The transfer roll 312 may comprise a roller with vacuum holes and a plurality of teeth, to maintain a CD extensed elastomeric substrate, such as a Back Ear or waistband stretch film, in a CD elongated disposition. It is to be appreciated that transfer roll 312 is only an example. A roller need not be employed. Instead, transfer roll 312 any be machine element holding the third substrate 103 extensed, such as a vacuum conveyor (not shown).

The preheating described in any of the embodiments described herein may be as simple as an air jet impinging upon one or more webs proximate a sonotrode. The distance of the hot air nozzle and associated hot air jet may be within about 10 mm, about 20 mm, or about 50 mm of the sonotrode. The hot air may be direct at a first, a second, or multiple substrates. Heating may be applied to first, second, or both sides of a nominally planar substrate. Where one substrate is on an anvil, hot air may be directed tangent to the surface of the substrate in a CD or circumferential direction or any tangent line intermediate. A guide may be used adjacent to the roll and, in some embodiments, conforming to the roll's surface with a spacing, such that an air jet may flow upstream or downstream along the substrate. The air jet may simply be adjacent such anvil roll, without a guide.

Heating may occur with one, two, or a plurality of substrates in free air at the heating point. Heating may be from a first side, a second side, or both sides of a substrate. Heating may be applied to the first side of a first web, where the first side is placed in operative contact and bonded to a second web. Heating may be applied to a second side of a first web, where a first side of said first web is placed in operative contact with and bonded to a second web. Heating substrates in free air may inhibit the web from cooling due to contact with an anvil roll, an anvil element (where a rotating anvil roll is not used), and/or a sonotrode. Heating in free air proximate the sonotrode may enable the temperature of a substrate to be as high as practical without melting.

Air flow may be in an MD or CD direction along a substrate. Where the substrate is air permeable, airflow may be through the substrate. A least a component vector of the hot air velocity may be normal to a nominally planar substrate. A hot air box may be formed upstream of the nip point. A nozzle on one side of a substrate may be used to apply hot air. A second nozzle on a second side may be used to extract air which has been partially cooled by heat transfer to a substrate. The temperature of preheat air may be near a softening temperature of a substrate. Higher preheat air temperatures may drive faster convective heat transfer, and enable higher line rates, but may result in deleterious process effects. Hot air temperatures near or above the melting temperature of one or more substrates may result in burn thru or holes, may result in wrinkles in the substrate, and/or may create thermal bonds at adjacent fibers. Thermal bonds between fibers in a nonwoven may result in a stiffer nonwoven, which may be stronger but may result in a harsh feel, which may be aesthetically unpleasing and undesirable.

Preheating airflow may be directed upstream along a substrate's MD from proximate a sonotrode. The highest temperatures may thus be proximate the bonding process, such that intermediate higher temperatures are avoided. The effect may be analogous to a counterflow heat exchanger. The partially cooled air travels upstream along at least one surface of a substrate. A nozzle with internal baffles may be created to directed hot air from a heat source towards the sonotrode nip point, and then divert the hot air flow upstream. A portion of the hot air flow may be deliberately diverted to impinge downstream towards the sonotrode. A heat shield may protect the sonotrode and/or anvil from overheating. An insulation layer may be applied to the sonotrode, which may simply be a non-conductive member such as a thick paper or fibrous mat. The sonotrode may be actively cooled, such as by a cold air blast or a conductive cooling. The sonotrode may have vents for cooling fluids, such as air, glycol, or heat spreaders. The hot air may heat the sonotrode to an elevated temperature, which may improve bonding. The anvil may also be heated.

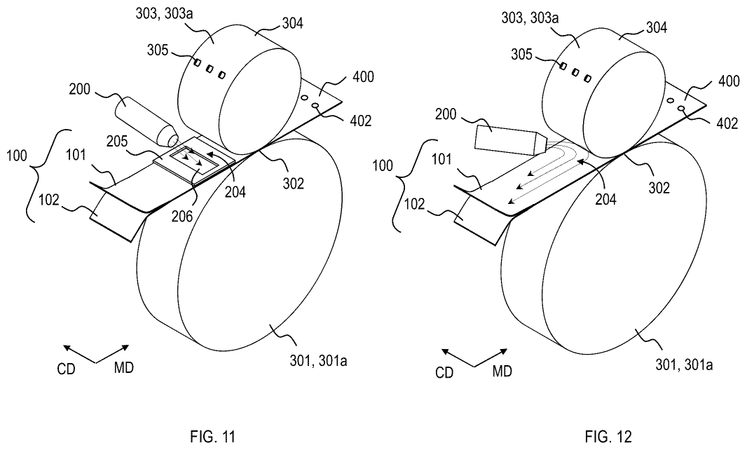

FIG. 11 is a schematic process flow diagram illustrating a method and an apparatus for bonding a plurality of substrates 100 that includes preheating a first substrate 101 of the plurality of substrates 100 employing a heat shield 205, comprising an aperture 206, and a cross directional CD flow of heated fluid 204. Heat may be applied to a region of a substrate, such as a CD strip, an MD strip, or a discrete region. For example, a heat shield may mask the substrate, to apply thermal energy to only a CD region of a product. A mask element moving with a substrate may direct heat to a bonding region. For example, an endless belt with a window corresponding to a bond site may direct heat to a region of a substrate, such as a region for a side seam bond. In some embodiments, heating may be applied in discrete regions, such as strips. Such masking may be arranged as a static heat shield which allows hot air to impinge on a substrate only in defined regions. In some embodiments, such a mask may be an endless belt with cutouts in discrete regions. The masking element may not travel through the bonding nip. The heated fluid 204 may pass through the aperture 206 to contact one or more of the plurality of substrates 100 before the plurality of substrates 100 pass through a nip 302 formed between an anvil 301 and a bonding device 303. The anvil 301 may be a first drum 301a. The bonding device 303 may be a second drum 303a. The second drum 303a may comprise an outer surface 304 and a plurality of projections 305 disposed on the outer surface 304. The plurality of projections 305 may form bonds 402 in the plurality of substrates 100 to form a bonded substrate 400.

FIG. 12 is a schematic process flow diagram illustrating a method and an apparatus for bonding a plurality of substrates 100 that includes preheating a first substrate 101 of the plurality of substrates 100 employing a partially cross directional flow CD of heated fluid 204 that is redirected in a counter machine direction MD. FIG. 12 is identical to FIG. 11 except that no heat shield 205 is employed. The heated fluid 204 may be directed at the nip 302 and may be reflected off of the anvil 301 and/or off of the bonding device 303 to be redirected in a direction that is substantially or partially opposed to the machine direction MD. Such a redirection may be desirable, because the heated fluid 204 is at its hottest at a point very close to the nip 302 and the redirected fluid 204 may still be able to warm one or more of the plurality of substrates 100 before they enter the nip 302, thereby conserving energy.

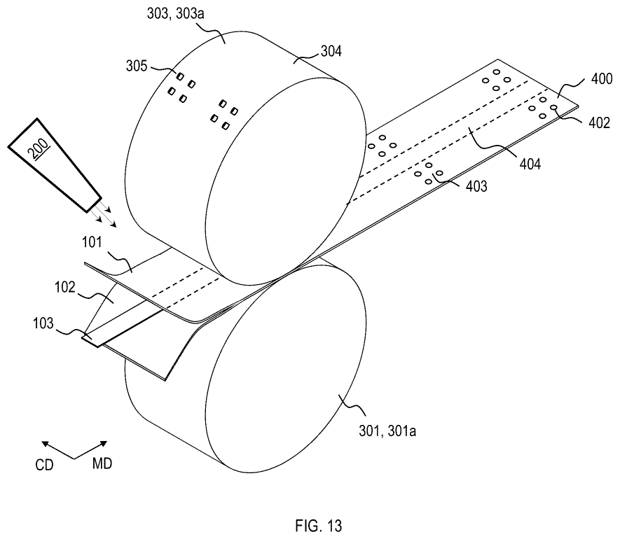

FIG. 13 is a schematic process flow diagram illustrating a method and an apparatus for bonding a plurality of substrates 100 as well as details of the bonded substrate 400. FIG. 13 is similar to FIG. 11 and FIG. 12 but includes a third substrate 103, which may be any type of substrate, but may preferably be an elastomeric material to be sandwiched between the first substrate 101 and the second substrate 102, which may be any type of nonwoven or film. The third substrate 103 and the projections 305 on the outer surface 304 of the bonding device 303 may be positioned such that the bonded substrate 400 comprises both a bonded area 403 and an unbonded area 404. The bonded area 403 may comprise bonds 402 while the unbonded area 404 may be free or substantially free of bonds 402. It may be desirable for some applications for the third substrate 103 to be partially, substantially, or completely disposed within the unbonded area 404.

Various embodiments relate to a laminate material, such as a bonded substrate 400, for an absorbent article. The laminate material may comprise a first substrate 101, such as a non-air-through bonded nonwoven material comprising fibers. The laminate may further comprise a second substrate 102 in a facing relationship with the first substrate 101. A plurality of bonds 400 may join the substrates 101, 102. The bonds 400 may individually or collectively form a bonded area 403. The bonds are or may be free of adhesives. The bonds are or may be ultrasonic bonds. The bonds may be pressure bonds. Unbonded area 404 may be defined between bonded areas 403. The unbonded area 404 may have at least 5 fibers per square inch that are fused at fiber-to-fiber intersections caused by preheating the nonwoven material, i.e., the first substrate 101. The second substrate 102 may be a nonwoven material. The second substrate 102 may be a film or an elastomeric film. Additionally or alternatively, a third substrate 103 may be included in the laminate. The third substrate 103 may be a nonwoven, a film, or an elastomeric film. The elastomeric films may be preactivated before being bonded. Various embodiments relate to an absorbent article comprising the laminate materials described herein. The absorbent article may, for example, be a taped diaper or a pant.

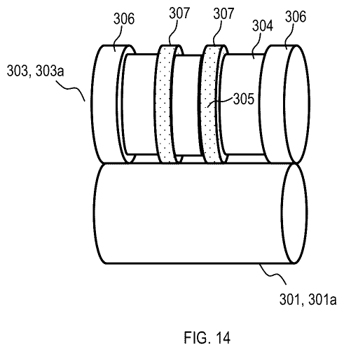

FIG. 14 is a schematic diagram illustrating a bonding device 303 in the form of a second drum 303a, having bearer rings 306 and pattern rings 307 on an outer surface 304 thereof. The pattern rings 307 may comprise a plurality of projections 305, which may be disposed to engage a product. The bearer rings 306 may set the clearance of interference of the pattern rings into an anvil roll 301. The bearer rings 306 may be disposed outside the substrate region, such that they do not engage the product material. The bearer rings 306 may be helpful when it is desired to create both bonded areas 403 and unbonded areas 404. Various configurations of an apparatus for compression bonding of thermoplastic substrates may depend on high compression rates and high compression forces to generate a flow of material from a central region of a bond site to an edge region of a bond site. Control of the thickness of the resulting membrane, and hence control of the amount of material flow to the edge of a bond site may be critical to forming reliable bonds. Such devices may comprise a first and second roll. At least one of such rolls may have a pattern of protruding nubs to form bond sites. A first roll may have substantially continuous rings, commonly referred to as bearer rings, contacting a second roll in a region where no substrate is presented. Such bearer rings may have a radial height slightly higher or slightly lower than the protruding nubs, to form a controlled high precision gap between nubs and anvil, thus setting a membrane thickness in the bond site, which may be less than the void free volume of substrate in the area defined by the nip. Bearer rings may have a continuous width or a variable width around the circumference of a roll. In some embodiments, the radial height of the bearer rings may be chosen to be the same or a small amount, say <2%, more than the radial height of the nubs to create a controlled overhang. Due to deflection of nubs and bearers, such overhang or under-hang controls the membrane thickness. Due to softening of substrates via preheat, bearer rings in a compression bonding with substrate preheat may be more critical than in the related art. Without bearer rings, preheated substrates may be more likely to experience holes at the bond site, which may weaken the bond or lead to equipment damage from metal to metal contact.

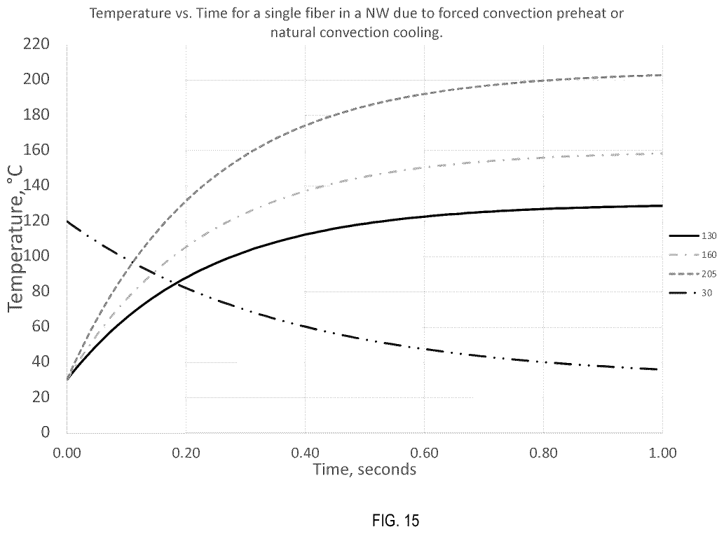

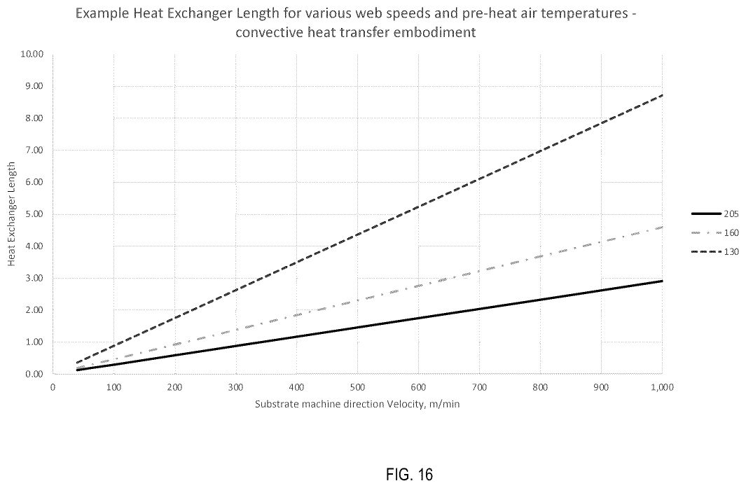

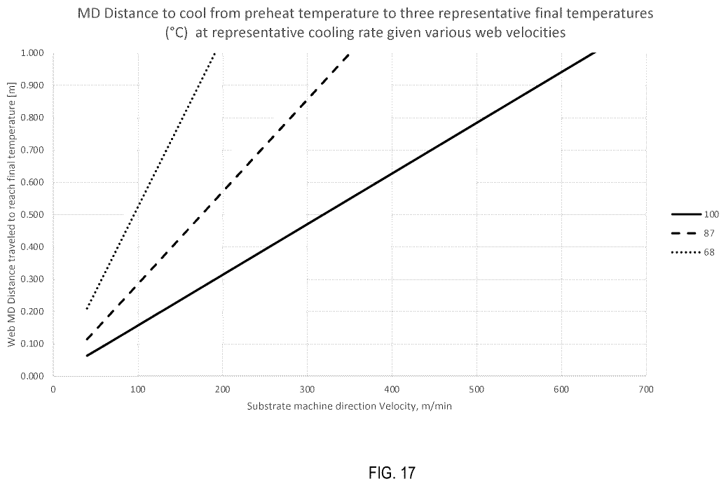

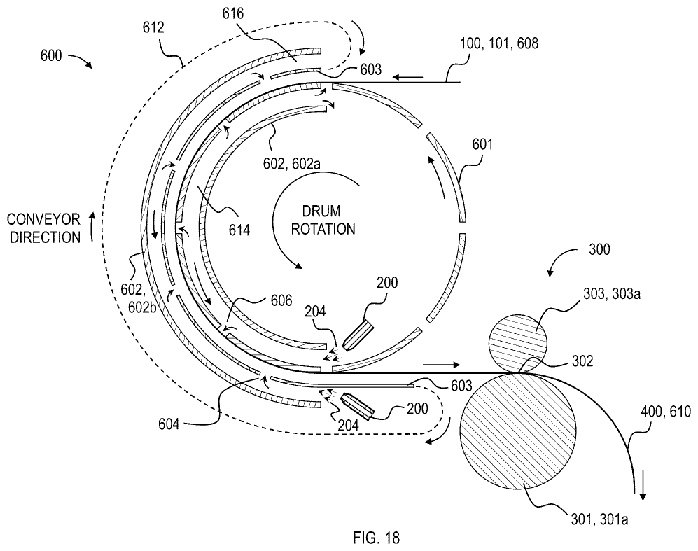

FIG. 15, FIG. 16, FIG. 17, and FIG. 18 cooperate to illustrate various thermodynamic considerations relevant to preheating relevant substrates. Without being limited by theory, preheating may lower the elastic modulus of a material, making the material softer. While mechanical compression and shear stresses may heat a polymer, such effects may be non-uniform. An advantage of this method is to preheat the substrate in a nonwoven form, where it is comprised of thin fibers. The characteristic required conductive heat transfer path from outer surface to center of the fiber may thus be short, and the fiber may heat relatively quickly and more uniformly than in other methods. The duration of the applied convective heat transfer via hot air may be chosen such that the fiber is substantially through heated to a target softening temperature. The temperature of the applied preheat working fluid may be chosen to eliminate overtemperature conditions on the outer surface of the fibers. Once the fiber is compressed by the compressive bonding roll and/or sonotrode, the result is a substrate that is at an elevated working temperature as an initial condition, with the applied compressive and/or shear energy urging a flow and/or further heating.

FIG. 15 is a chart plotting temperature vs. time for a single fiber in a nonwoven due to forced convection preheat at various air temperatures or natural convection cooling.

FIG. 16 is a chart plotting heat exchanger length for various web speeds and pre-heat air temperatures for a convective heat transfer embodiment.

FIG. 17 is a chart plotting cooling Length for various web speeds to reach a final temperature at representative cooling rates.

To provide sufficient preheat, a substrate must be exposed to a hot air source for sufficient duration for thermal energy to transfer from the heated air to the surface of the substrate and further to transfer the thermal energy via conduction from the outer surface through the central region of a substrate. FIG. 15 and FIG. 16 provides an example of a round polypropylene fiber of 20 micron diameter in an airstream with a convective heat transfer coefficient of 40 W/(m²·K). The required preheat duration in the time domain FIG. 15 and the position domain is provided in FIG. 16. This example is not intended to be limiting, and a low heating rate of 40 W/(m²·K) is chosen as a simple low heating rate estimate for forced convection using an easily achieved 15 m/s airstream. It is to be appreciated that higher heat transfer coefficients, and thus shorter preheat configurations may be achieved with different heating geometries, higher air velocities, higher pre-heat temperatures, and/or more turbulent airflows.

The preheat may comprise a first order linear time invariant system, with a time constant driven by the thermal diffusivity. The time constant may be calculated from the thermal diffusivity and characteristic conduction path length, L_{c}. The characteristic time constant is n·L_{c2}/(thermal diffusivity), where n is the number of time constants. It is desirable to have at least one time constant, two time constants, or three time constants of heating duration available for preheat. These correspond to a time of 1·L_{c2}/(thermal diffusivity, 2·L_{c2}/(thermal diffusivity, and 3·L_{c2}/(thermal diffusivity) respectively. These time constants may be converted to an MD length dimension by multiplying the time constant by the lineal substrate velocity in the MD. For film substrates, the characteristic heat flow path L_{c} is defined for this use as a film thickness for a film heated on one side or a half the film thickness for a film heated on two sides. For air permeable nonwoven substrates, the L_{c} for this use case is simplified to that of the characteristic fiber diameter. The L_{c} is approximated as the fiber radius for nominally cylindrical fibers. For non-cylindrical fibers, the characteristic heat flow path may be calculated as the mean depth from the surface of the fiber to the centroid of the fiber shape. The preheat system may be approximated as a linear time invariant system. The preheat may achieve 63%, 86%, and 95% of the difference between the initial substrate temperature and the applied preheat air temp in 1, 2, and 3 time constants respectively. For the small fiber diameters typical of nonwovens, additional pre-heat duration may not be useful. For a nonwoven or laminate comprising a plurality of nonwoven fibers, some of the fibers, melt-blown layers in the nonwoven or laminate may impede airflow to other parts of the substrate. Additional pre-heat may be required to heat soak all regions of a substrate with heating. Preheat temperatures above the intended substrate temperature at bonding may be used to reduce the required preheat duration.

For example, a polypropylene with 162° C. melting point intended to bond at 130° C. may be preheated by hot air at 160° C. to achieve a faster preheat. The example substrate may be heated by hot air at a temperature above the substrate's melting temperature, such as 205° C. in the example. Arranged properly, the substrate may not melt, but may reach an intended preheat temperature more quickly. In some cases, multiple preheat stages may be utilized. Continuing the example of a 30° C. PP heated to 140° C. by 205° C. preheat air, the preheat of 90° C. via an air stream at 175° C. above the initial substrate temperature may be arranged to occur in about 1 time constant. After the initial preheat, a second preheat zone, for example 140° C. in this case, may be used to keep the substrate at the intended bonding temperature while it is transported into the nip point. Preheat may be accomplished in a shorter duration by improving the rate of heat transfer. For example, some industrial preheaters work at a slow air velocity, such as 5 m/s for through air heating where the airflow may be orthogonal to the plane of the substrate. For ultrasonic and high force compression bonding of thermoplastic substrates, a higher air velocity of 15 m/s or high may be desired. Many absorbent article component substrates have reduced or negligible air permeability, meaning air will not flow through the substrate or will not easily flow through the substrate. For example, diaper cuff materials typically have a melt-blown film layer, which is intended to block fluid transport of urine, feces, and water. In such cases, flow along a surface of a substrate rather than flow through a substrate may be used to transfer thermal energy to a substrate. In some cases, thermal energy may be applied to a first and second surface of a substrate concurrently. Higher air velocities may provide improved convective heat transfer from air to the substrates outer surface. Ensuring the airflow to the substrate surface is turbulent may often be used to improve the heat transfer. Laminar flow may result in a boundary layer of partially cooled air. Internal heat transfer through the substrate is still subject to conductive heat transfer. As thermoplastic polymers may have low thermal effusivity due to their high heat capacity and lower thermal conductivity, thinner fibers or films may be desired. Thermal effusivity is a measure of the ability of a material to exchange heat with its immediate surroundings at a surface.

For preheating of substrates prior to bonding, two heat transfer steps are required with the substrate. Thermal diffusivity (alpha) is k/ρ·cp),

where
k is thermal conductivity (W/(m·K))
cp is specific heat capacity (J/(kg·K))
ρ is density (kg/m³)

Together, ρcp can be considered the volumetric heat capacity (J/(m³·K)).

The time constant for conductive heat transfer through a planar substrate may be calculated from F0(T)=(thermal diffusivity)(time constant)/(conductive path length)². For the first time constant, we solve for the time constant as 1−((conductive path length)²)/(thermal diffusivity). This simple model applies for a film substrate. Note that the conductive heat flow path length is the film thickness if heating one side of the substrate but only half the film thickness if heating of both sides of the substrate is implemented.

For heating of a fiber, a similar principle applies. As a simplifying approximation, the above planar solution may be applied, such that where the heating air impinges around the fibers, the characteristic heat flow length will be treated as approximately half the fiber diameter for a round fiber. It will be obvious to one skilled in the art that the conductive heat transfer time constant will be slightly reduced for fibrous substrates where the fiber provides a higher surface to volume ratio than a planar film substrate. The differently, a cylindrical fiber of radius R has only 25% of its volume within an inner radius (R/2) and 75% of its volume between R/2 and R. The average heat flow path length on a volumetric basis from outer surface of a cylindrical fiber of Radius R to the 0.707R is thus about 0.29R.

For a typical 20 micron fiber, heating simulations show less than 5° C., less than 2° C., and less than 1° C. temperature difference from the outside to the central region of a cylindrical fiber. In another example, a 60 micron diameter fiber had less than 0.5° C. temperature difference from perimeter to center. One of the key advantages of the hot air preheating method may thus be to heat the central region of the fiber and the central fibers in a nonwoven. Other heating methods, such as the heating which occurs due to plastic flow, may only heat a portion of the fiber. In some cases, compression heating may only affect an outer region of a fiber, with a central region still relatively cold at a high modulus, with significant resistance to flow.

Conversely, cooling is the inverse of the preheating process. Cooling of even 5° C. may have an adverse effect on bond strength from ultrasonics or compression bonding. The ideal may be to arrange the system such that the substrate does not cool between the preheat and the bonding nip. An embodiment may be to have the hottest preheat air adjacent the nip point. In some embodiments, a first and second preheat air source may be used. For example, preheat air at T_PH1 may be applied upstream with a curtain of air at T_PH2 continuing from the first preheat region to a second preheat region. The first preheat region may be used to quickly heat the substrate and the second preheat region utilized to limit or inhibit cooling of the substrate. The second preheat region may include a blanket of hot air near the intended substrate temperature at bonding.

As shown in the FIG. 17, an example polypropylene nonwoven at 120° C. form a pre-heat operation, figures “Cooling length” provide an example of the short MD distance required to cool off to 100° C., 87° C., and 68° C. At a slow velocity of 700 feet/min (213m/min), even 300 mm (˜1 ft) of distance will result in 20° C. of cooling. While higher line speeds provide less time for cooling, such line speeds may also accelerate cooling by providing a higher convective heat transfer cooling rate due to increased air velocity relative to the substrate. Such higher web speeds are also more likely to result in localized turbulent flow.

FIG. 18 is a schematic process flow diagram illustrating a method and a device 600 for preheating in association with a machine 300 for bonding. In general, the preheating of a substrate according to any of the embodiments described herein may occur on a drum. A drum may simply be a heated roll, with conductive heat transfer to a substrate. A drum may be air permeable, with a surface which supports the web without blocking air. A mesh or honeycomb material may be used for this purpose. Hot air may be directed to a central region of a drum. The central region may comprise a sector of the drum. An external hood may be used outside the drum to collect the air. Such designs may be reapplied from through air drying technology. The collected air in this drum concept of any of the preceding concepts may be recirculated. The airflow through the drum may be reversed, with flow from external the drum to inside the drum. More specifically, the method and device 600 may be employed to preheat one or more substrates 100, 101 or an unbonded composite structure 608 and to form a bonded substrate 400 or a bonded composite structure 608. The composite structure 608 may be, for example, a connected series of unbonded pant-style diapers or any other multi-layer or multi-component structure. The preheating device 600 may comprise a rotatable drum 601 and a conveyor 603 cooperatively operating to convey the one or more substrates 100, 101 or unbonded composite structure 608 toward the bonding machine 300. The bonding machine 300 may be configured as described in any of the other embodiments and is not limited to the specific structures shown. In general, the bonding machine 300 may comprise a nip 302 defined between a bonding device 303 and an anvil 301. The preheating device 600 may convey the one or more substrates 100, 101 or the unbonded composite structure 608 to the nip 302. The rotatable drum 601 may comprise a plurality of apertures 606 in an outer surface thereof through which heated fluid 204 from a heating apparatus 200 may flow to impinge upon and to heat the one or more substrates 100, 101 or the unbonded composite structure 608. Similarly, the conveyor 603 may comprise a plurality of apertures 604 in an outer surface thereof through which heated fluid 204 from a heating apparatus 200 may flow to impinge upon and heat the one or more substrates 100, 101 or the unbonded composite structure 608. The apertures 606 on the drum and the apertures 604 on the conveyor 603 may be aligned or offset relative to each other. One or more heat shields 602 may be employed to direct air toward the apertures 606, 604. An internal heat shield 602a may be disposed within the drum and may maintain a fixed position while the drum rotates. Similarly, an external heat shield 602b may be positioned within the bounds of a path 612 defined by the conveyor and may maintain a fixed position while the conveyor moves. An internal channel 614 may be defined between the internal heat shield 602a and the rotatable drum 601 through which heated fluid 204 may be directed toward the apertures 606 of the drum 601. Similarly, an external channel 616 may be defined between the external heat shield 602b and the conveyor 603 through which heated fluid 204 may be directed toward the apertures 604 of the conveyor 603.

While CD oriented stretch is the driving consideration, the method is re-applicable and useful in multiple applications. For example, the methods are applicable to elastic laminates with CD stretch, MD stretch, and elastomeric elements at various angles in between. The method is applicable to elastomer films, elastomeric nonwovens, and stranded elastomeric elements. An example of MD stretch laminates is the elastic belt common on pants diapers. Pants diapers commonly feature first and second nonwovens with stranded elastics laminated intermediate via a hot melt adhesive. Other embodiments of pants diapers may comprise two nonwovens with an elastomeric stretch film laminated intermediate. Preheating and ultrasonics bonding may enable elimination or reduction of said glue or may enable a line rate increase, so such bonding is done on a laminator line separate from a diaper converter.

The methods are applicable to the side seam of a first belt to a second belt in a pants diaper. Hot air may be applied to heat the side seam region, then ultrasonic compression energy applied via a blade or rotary sonotrode to bond a first and second stretch belt together. Such heating may occur on a drum. As shown in FIG. 18, a drum rotates with two extensible belt laminates on its outer surface, the laminates connected by a diaper core. A region of a first or second belt laminate may be heated via hot air. The drum may have a plurality of windows, which may comprise mesh elements, to direct hot air to preheat a substrate. The outer surface of the drum may have hot air, which may heat a first belt. A belt, which may be endless, with windowed holes or other masking device may direct the hot air to preheat regions of the second belt.

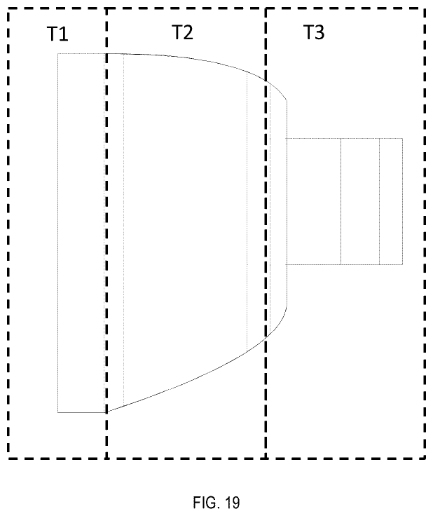

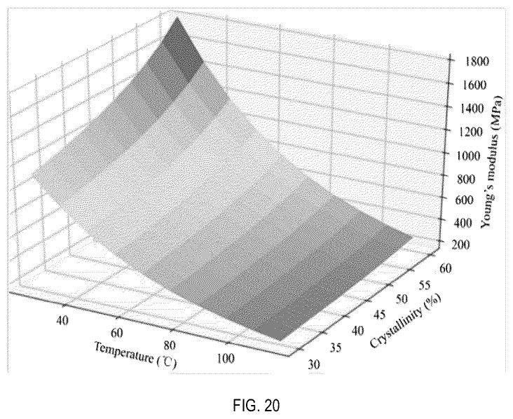

FIG. 19 is a schematic diagram illustrating a various preheating zones for a back ear of a diaper. Various embodiments may provide a line rate increase of offline back ear lamination machines for making back ear laminates as shown in FIG. 20, which may be used in various absorbent articles. Online machines may run slowly, for examples, 80-120 m/min or under 150 m/min. Offline machines may run higher speeds, which may run from 300 to 400 m/min. Such high speed lines may require special anvils with a high density of bond sites, may have a small process window, and may be unreliable for a plurality of reasons including equipment failures of the ultrasonic system. The ultrasonic bond strength of back ear or waistband laminate at such line speeds may be inadequate. By applying heat proximate the bonding nip between sonotrode and anvil, the strength of the bonds was greatly increased. Even a low substrate preheats to 60° C. made a substantial improvement in nonwoven-nonwoven bond force, enabling speeds of 500 m/min and likely higher to meet quality requirements. For pure nonwoven-nonwoven bonds, the substrates are heated to a temperature intermediate the melting temperature and a softening temperature. For example, a polypropylene nonwoven may have a melting temperature of 162° C., a softening temperature of 90-150° C., and a preheat temperature of 100-140° C. As the substrate may cool quickly, heat may be applied as close as practical to the nip point. A portion of the hot air may be directed along a substrate towards the nip point, to provide a blanket of hot air and reduce cooling intermediate preheat and nip.

In stretch laminate applications such as ultrasonic ear laminate, the pre-heat temperature may be limited by thermal degradation of an elastomeric or stretch component in the laminate stack-up. For example, preheat to 100° C. may effectively destroy the stretch properties of SBC (styrenic block co-polymer) film used in some laminates. In the case of an ultrasonic bonded elastic laminate film, the SBC film may comprise a laminate of a polyolefin skin (e.g., 1-3 micron PP) laminated to an SBC core (e.g., ˜40-50 microns thick). Skins may be on a first or first and second sides of a stretch film. A simple method of inhibiting damage to the stretch film may be to choose an intermediate temperature. For example, testing shows limited film degradation at 50° C. and substantial film degradation at 100° C. A process setpoint may be chosen between these limits. In other embodiments, the thickness of the skin layer may be increased, to limit the thermal degradation of the elastomeric substrate. The chemistry of the skins or SBC core may be changed to limit thermal degradation. For example, a PP variant with a higher melting temp or a higher crystallinity percentage may be chosen. The stretch film may be replaced by an elastomer which is not sensitive to temperature below the melt temp of the nonwoven. For example, some elastics, which may include LYCRA® (also known as, elastane, a polyether-polyurea copolymer), may be formed with a solvent and thus less sensitive to heat. Replacing the CD stretch film with stranded elastics oriented in a CD direction may enable higher line rates. Stranded elastics may enable a more breathable product. The preheat temperature may be varied in zones, or regions. Such regions may be across a CD direction. Such regions may be defined by a mask or heat shield. Such masks may move with the substrate or be fixed. For example, the distal nonwoven-nonwoven or nonwoven-nonwoven-nonwoven bond region of ultrasonically bonded stretch laminate may have a high bond force requirement, whereas the medial nonwoven-stretch film-nonwoven region may have a lower bond force requirement. An inner nonwoven-nonwoven region of the laminate may have a high bond force requirement. Said differently, the bond force requirement may be lower in the corrugated region of an ultrasonically bonded stretch laminate back ear, as most force are through the film and the nonwoven is an aesthetic cover, whereas the film edge(s) must be securely anchored to nonwoven at the chassis and/or fastening tape. A first preheat temperature may be used in one region, and a second preheat temperature in a second region. A plurality of preheat regions may be utilized, and each may have its own temperature and airflow control. Preheat regions may be arranged by substrate. For example, a first nonwoven and a stretch film on a vacuum anvil may be heated to a first temperature, such as 50° C., which is below the thermal degradation temperature of the stretch film. A second nonwoven may be preheated to a second temperature, such as 115° C. The preheat may enable improved nonwoven flow, while the high thermal mass of the elastomer prevents the small thermal energy of the second nonwoven from damaging the elastomeric properties. Preheat need not be entirely via hot air. A patterned anvil roll may be heated, so that a first side of a first nonwoven on the pattern roll is at least partially preheated via conduction while a film positioned against a second side of the first nonwoven is only minimally heated. The second nonwoven may be heated via hot air and may be heated to a temperature higher than the first nonwoven. The plurality of materials may then be ultrasonically bonded.

In some embodiments, preheating may eliminate the requirement to apply ultrasonic vibrational energy. Instead, the combination of an elevated preheat temperature, lowering the material modulus, may enable a simple compression bond. Substrate preheating may enable bonding without the cost and complexity of ultrasonic equipment such as sonotrodes, actuators, generators, and controllers. Some substrates, such as an ultrasonically bonded elastic laminate structure, may be possible to bond with just a compression bond. Preheat may be combined with mechanical compression in a chosen range of shear rate. Compression rates may be chosen to be at least 1000 inverse seconds, 1e4, 1e5, 1e6 inverse seconds or higher.

FIG. 20 shows a representative response curve for PP over a range of temperature and crystallinity. A typical absorbent article nonwoven might start at 22° C. and about 50% crystallinity. By heating the nonwoven, the modulus may be reduced from about 1200 MPa in an example to about 200 MPa at 120° C. The degree of crystallinity may be reduced by the heating, which may be due to conversion of some crystalline domains to an amorphous state which flows. Polymers in the amorphous state may flow more readily and may reform crystalline domains once the polymer cools. Preheating may be chosen to induce a material state where the Young's modulus, or elastic modulus, of the material is reduced. Such reduction may be by a factor reduction factor of 20%, 50%, 100%, 200%, 400%, or even 600+%. This reduction in required compression force for a given bonded area may enable different patterns, more unbalanced patterns, or patterns with a greater bonding area. This process may enable bond patterns which form aesthetically pleasing patterns or insignia. The required compressive stress for high compression tooling may exceed the ultimate stress of mot materials. Preheating substrates may enable the use of alternate materials or may enable a compression only process to bond in new applications.

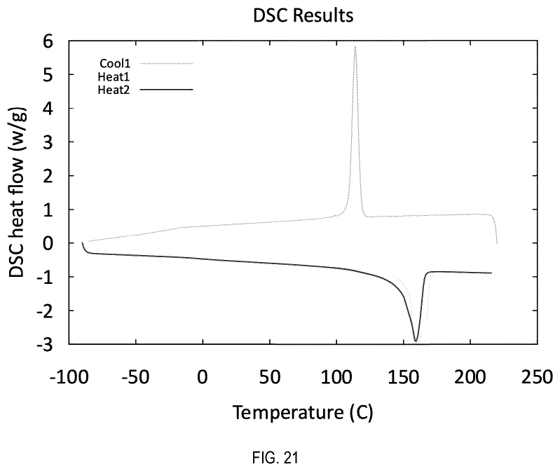

As shown in FIG. 21, preheat may be defined by differential scanning calorimetry of a substrate. For the example PP in the graph, a deviation of the heat flow rate in watts/gram is seen at about 100° C. Said simply, the heat flow rate is nominally linear up to this softening temperature, and then the slope of the heat flow begins to change. The example material fully melts at about 162° C. The graphic shows two complete heating cycles, with a first heating, a cooling back to a solid, and a second heating. The heat flow out of the material upon cooling shows a substantial spike between about 100° C. and 125° C. It may be advantageous to preheat to a temperature range just under, at, or just about this range as determined by the cooling portion of DSC. This cooling heat flow spike may correspond to recrystallization of domains in the polymer. As such, the crystallization process may be sensitive to cooling rates and sample size, among other parameters.

### Examples/Combinations

1. A laminate material for an absorbent article, comprising:
a non-air-through bonded nonwoven material comprising fibers;
a substrate in a facing relationship with the nonwoven material;
a plurality of bonds joining the nonwoven material to the substrate, wherein the bonds each form a bond area in the nonwoven material and the substrate; and
an unbonded area formed in the nonwoven material and the substrate, wherein the unbonded area is free of the plurality of bonds;
wherein the unbonded area in the nonwoven material has at least 5 fibers per square inch that are fused at fiber-to-fiber intersections caused by preheating the nonwoven material.
   2. The laminate material of Paragraph 1, wherein the substrate is a nonwoven material.
   3. The laminate material of Paragraph 1, wherein the substrate is a film or an elastomeric film.
   4. The laminate material of Paragraph 3, wherein the film is preactivated before being joined with the nonwoven material.
   5. The laminate material of any one of the preceding paragraphs, wherein the bonds are free of adhesives.
   6. The laminate material of any one of the preceding paragraphs, wherein the bonds are ultrasonic bonds.
   7. The laminate material of any one of Paragraphs 1-5, wherein the bonds are pressure bonds.
   8. An absorbent article comprising the laminate material of any one of the preceding paragraphs.
   9. The absorbent article of Paragraph 8, wherein the absorbent article is a taped diaper or a pant.
   10. A material for an absorbent article, comprising:
a non-air-through bonded nonwoven material comprising fibers;
a plurality of bonds formed in the nonwoven material, wherein the bonds each form a bond area in the nonwoven material; and
an unbonded area formed in the nonwoven material, wherein the unbonded area is free of the plurality of bonds;
wherein the unbonded area in the nonwoven material has at least 5 fibers per square inch that are fused at fiber-to-fiber intersections caused by preheating the nonwoven material.
   11. The material of Paragraph 10, wherein the bonds are free of adhesives.
   12. The material of Paragraph 10 or 11, wherein the bonds are ultrasonic bonds.
   13. The material of Paragraph 10 or 11, wherein the bonds are pressure bonds.
   14. An absorbent article comprising the material of any one of Paragraphs 10-13.
   15. The absorbent article of Paragraph 14, wherein the absorbent article is a taped diaper or a pant.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present disclosure have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method for bonding a plurality of substrates in a nip provided between an anvil and a bonding device, the method comprising:
preheating a portion of a first substrate of the plurality of substrates for a preheating duration to impart a preheated temperature to the portion;
conveying the plurality of substrates to the nip, wherein the portion of the first substrate reaches the nip at a final temperature that is within 0° C. to 40° C. of the preheated temperature; and
conveying the plurality of substrates through the nip to form a bond in at least some of the plurality of substrates, wherein a time-in-nip to create the bond is less than 10 milliseconds;
wherein the preheating duration is at least 200% longer than the time-in-nip.

2. The method of claim 1,
wherein the bonding device applies pressure to the plurality of substrates without the use of vibrational energy;
wherein the anvil is a first drum;
wherein the bonding device is a second drum comprising projections extending outwardly from an outer surface thereof; and
wherein the method comprises providing one or more bearer rings on the second drum.

3. The method of claim 2, wherein the bond is free of adhesives.

4. The method of claim 1, wherein the bonding device is an ultrasonic bonding device, and wherein the bond is free of adhesives.

5. The method of claim 4, wherein the anvil comprises a plurality of protrusions extending outwardly from an outer surface thereof.

6. The method of claim 4, wherein the ultrasonic bonding device is a blade sonotrode or a rotary sonotrode.

7. The method of claim 4, comprising cooling the ultrasonic bonding device.

8. The method of claim 1, wherein the plurality of substrates comprise a first substrate and a second substrate, wherein the first substrate is a nonwoven material or a film, and wherein the second substrate is a nonwoven material or a film.

9. The method of claim 1, wherein the preheating comprises directing a heated fluid at the first substrate.

10. The method of claim 9, comprising recirculating the heated fluid.

11. The method of claim 9, wherein the heated fluid has a temperature in the range of about 90° C. to about 300° C.

12. The method of claim 1, wherein preheating the portion of the first substrate comprises a plurality of stages, including at least a first stage and a second stage;
wherein the first stage comprises heating the portion of the first substrate for a first duration to impart a first temperature to the portion;
wherein the second stage comprises heating the portion of the first substrate for a second duration to impart a second temperature to the portion; and
wherein the first temperature and the second temperature are different.

13. The method of claim 1, wherein the preheating duration is about 50 milliseconds to about 200 milliseconds.

14. The method of claim 1, wherein preheating the portion of the first substrate is completed within a distance of about 5 mm to about 500 mm of the nip.

15. The method of claim 1, comprising preheating a portion of a second substrate of the plurality of substrates for a second preheating duration to impart a second preheated temperature to the portion of the second substrate.

16. The method of claim 15, wherein the portion of the first substrate comprises a surface thereof;
wherein the portion of the second substrate comprises a surface thereof; and
wherein the portion of the first substrate and the portion of the second substrate are in a facing relationship when the plurality of substrates are conveyed through the nip to form the bond.

17. The method of claim 1, wherein the plurality of substrates comprises the first substrate, a second substrate, and a third substrate;
wherein the first substrate comprises a nonwoven material;
wherein the second substrate comprises a nonwoven material;
wherein the third substrate comprises an elastic film; and
wherein the elastic film is displaced from the bond.

18. The method of claim 1, wherein the anvil and/or the bonding device is heated, wherein the portion of the first substrate comprises a substantial portion of a thickness thereof, and wherein conveying the plurality of substrates through the nip occurs at a speed greater than 250 m/min.

19. A method of bonding substrates, comprising:
providing a machine comprising an anvil and a bonding device;
providing a nip between the anvil and the bonding device;
conveying a first substrate and a second substrate toward and through the nip;
upstream of the nip, preheating the first substrate or the second substrate; and
bonding the first substrate to the second substrate in the nip to create a bond;
wherein time-in-nip to create the bond is less than 20 milliseconds;
wherein a duration of the preheating step is at least 200% longer than the less than 20 milliseconds; and
wherein the conveying occurs at a speed greater than 300 m/min.

20. A method of bonding substrates, comprising:
providing a machine comprising an anvil and a bonding device;
providing a nip between the anvil and the bonding device;
conveying a first substrate and a second substrate toward and through the nip;
upstream of the nip, preheating the first substrate or the second substrate;
bonding the first substrate to the second substrate in the nip to create a bond;
wherein time-in-nip to create the bond is less than 10 milliseconds;
wherein a duration of the preheating step is at least 200% longer than the less than 10 milliseconds; and
wherein the preheating comprises heating a substantial portion of the thickness of the first substrate or the second substrate.
